(19) United States
(12) Patent Application Publication
(10) Pub. No.: US 2007/0149490 A1
(43) Pub. Date: Jun. 28, 2007

(54) PROCESS FOR THE EXTRACTION OF BETULINIC ACID

(76) Inventors: Carsten H. Puder, Ingelheim (DE); Herbert Graef, Ober-Hilbersheim (DE); Markus J. Thumerer, Mainz (DE); Markus Heitzmann, Ingelheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/538,124

(22) Filed: Oct. 3, 2006

(30) Foreign Application Priority Data
Oct. 12, 2005 (EP) 05109482

(51) Int. Cl. A61K 31/56; C07J 53/00

(52) U.S. Cl. 514/169; 552/511

(57) ABSTRACT

Disclosed are large-scale industrial processes for obtaining highly pure betulinic acid from ground plane bark. Betulinic acid is 3β-hydroxy-lup-20(29)-ene-28-oic acid of formula

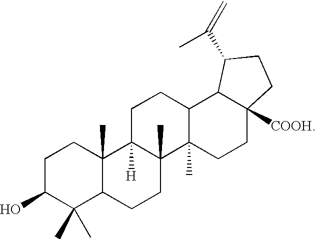

### APPLICATION DATA

This application claims benefit to EP05109482.9 filed Oct. 12, 2005.

### BACKGROUND TO THE INVENTION

The invention relates to an industrial-scale process for obtaining highly pure betulinic acid from ground plane bark. Betulinic acid is 3β-hydroxy-lup-20(29)-ene-28-oic acid of formula

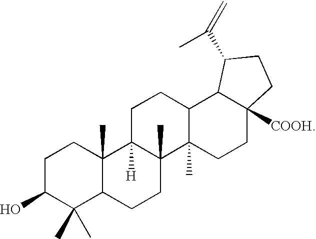

It is known that betulinic acid has an effect against the growth of melanoma cells (e.g. Pisha et al., Nature Medicine 1, 1995, 1046 ff) as well as against other cancer cells (e.g. Sunder et al., U.S. Pat. No. 6,048,847). In addition its derivatives are supposed to be capable of being used to combat HIV (e.g. Evers et al., J. Med. Chem. 39, 1996, 1056 ff; Soler et al., J. Med. Chem. 39, 1996, 1069 ff or Butler, Nat. Prod. Rep., 2005, 22, 162 ff). Consequently there is great demand for betulinic acid.

Apart from synthetic production (e.g. L. Ruzicka et al., Helv. Chim. Acta 21, 1938, 1076 ff) betulinic acid may also be obtained from various plants, particularly trees, such as for example from the bark of Picramnia pentandra (e.g. Herz et al., Phytochemistry 11, 1972, 3061 ff), from the bark of Arbutus menziesii (Robinson et al., Phytochemistry 9, 1970, 907 ff) and from the bark of Ziziphus mauritiana (e.g. Pisha et al., Nature Medicine 1, 1995, 1046 ff).

However, it is difficult to isolate the betulinic acid from these starting materials. A more promising prospect is its recovery from the bark and/or cortex of the plane tree. DE 197 13 768 proposes a method of recovering betulinic acid in which a powder obtained from plane cortex is extracted with a medium-polarity solvent, such as for example dichloromethane, chloroform or diethyl ether.

This process is, however, unsuitable for the industrial recovery of large amounts of betulinic acid, as very large volumes of the medium-polarity solvents have to be used to extract the betulinic acid (7 litres of dichloromethane, to 150 g of powdered plane bark). Moreover, extraction carried out in a glass column under hydrostatic pressure is not suitable for large amounts and presents problems from the point of view of safety.

Bruckner et al., J. Chem. Soc. 1948, 948-951 describe a process for obtaining betulinic acid from plane bark in which the ground-up bark is extracted with methanol, the extract obtained is evaporated down and the concentrate is repeatedly crystallised from methanol in the presence of charcoal. However, the betulinic acid thus obtained still contains a variety of contaminants.

WO 03/066659 discloses a process for obtaining betulinic acid from a methanolic extract of ground plane bark. The yield is 2.3% in crude form, based on the plane bark used, but does not have the desired purity, even though an additional purification step is carried out which comprises stirring the extract with n-hexane.

The aim of the present invention is therefore to provide an improved process for obtaining betulinic acid from plane bark, which can also be used on an industrial scale.

### DETAILED DESCRIPTION OF THE INVENTION

Surprisingly it has now been found that the conversion levels can be significantly increased using the process according to the invention. Furthermore, the amount of waste can be kept low by avoiding the use of chromatography materials.

Accordingly, the present invention is based on an improved process for obtaining highly pure betulinic acid by extracting ground plane bark and/or cortex, which is characterised in that the extraction is carried out with a solvent mixture consisting of toluene, xylene or toluene/xylene, preferably toluene or xylene and
one or more C₁₋₆-alcohols, or
esters of acetic acid, or
C₃₋₆-ketones.

It is particularly preferable to use a solvent mixture of toluene or xylene and
methanol, ethanol, n-propanol, 2-propanol, n-butanol, t-butanol, n-pentanol, (+−)-2-pentanol or 3-pentanol, or
methyl acetate, ethyl acetate, 2-propyl acetate, or
acetone, methylethylketone or methyl-2-butylketone.

A preferred solvent mixture consists of toluene and two alcohols selected from among methanol, ethanol, n-propanol, 2-propanol, n-butanol, t-butanol, n-pentanol, (+−)-2-pentanol or 3-pentanol. Particularly preferably the solvent mixture consists of toluene, methanol and a second alcohol (ethanol, n-propanol, 2-propanol, n-butanol, t-butanol, n-pentanol, (+−)-2-pentanol or 3-pentanol), particularly preferably of toluene, methanol and (+−)-2-pentanol. In a particularly preferred variant of the process the solvent mixture used can be recovered very easily, thus minimising the loss of solvent in the recycling process; toluene in a ratio of between 80:20 and 95:5 to the above-mentioned alcohols has proved particularly suitable for this purpose.

Particularly preferred is the above process wherein the plane bark is moistened with water before the extraction. On the one hand this can improve the workability of the plane bark and on the other reduce the environmental dust pollution caused.

The process described above may be carried out continuously or as a batch process, but the above process in which extraction is carried out continuously is preferred.

Preferably the process is carried out continuously with a flow rate of the solvent mixture of 0.2 m³/h to 6 m³/h within a period of 2 h to 48 h, preferably 5 h to 35 h, particularly 15 h to 25 h. Battery extraction equipment of variable size and construction may be used. The average daily amounts of powdered plane bark for extraction may be between 500 kg and 8 tonnes.

The above process wherein the extraction is carried out at a temperature of 20 to 110° C., preferably 40 to 90° C., particularly preferably 50 to 70° C., is particularly preferred.

For obtaining the betulinic acid from the crude extract the process is followed by some additional steps. Therefore, the above process wherein the following steps successively for isolating the betulinic acid are carried out is preferred:
a) extraction of plane bark in a solvent mixture according to one of claims 1 to 4;
b) removal of the solvent mixture from the extract;
c) dissolving the residue in a suitable solvent;
d) precipitating betulinic acid from the filtrate.

Suitable solvents in step c) may be mixtures of C₁₋₄-chloroalkanes or acetic acid esters and C₁₋₄-alcohols. Preferred chloroalkanes are dichloromethane and chloroform, preferred acetic acid esters are methyl acetate, ethyl acetate, 2-propyl acetate and preferred alcohols are methanol, ethanol and 2-propanol. Particularly preferred is a mixture of dichloromethane and methanol in ratios of 5:1 to 1:2, preferably 3:1 to 1:1, particularly preferably 2:1 to 1.5:1. A suitable amount of solvent or solvent mixture is used, based on the solid residue. A preferred ratio is 5 to 30 litres, particularly preferably 16 to 20 litres per kilogram of solid. The precipitation of the betulinic acid is preferably carried out by slowly cooling the filtrate to −15 to 10° C., preferably −10 to 10° C., particularly preferably 0 to 10° C. within a period of 5 to 50 h, preferably 5 to 20 h, particularly preferably 10 to 15 h. Then the purified betulinic acid may be separated from the mother liquor as a solid and dried using methods known in the art (suction filter, centrifuge, decanter, pressure filter, filter dryer etc.). The betulinic acid thus purified may still contain up to 10% solvent.

Another intermediate step can substantially increase the purity of the betulinic acid and make further processing easier. In a preferred process, the following steps are carried out between steps b) and c):
b1) suspending and dissolving the residue in propanol, butanol or a mixture thereof;
b2) combining with a filtering or suction composition
b3) filtering the suspension
b4) precipitating the crude betulinic acid from the filtered extract.

Steps b1) to b4) may optionally be carried out once or several times. Preferably the suspending in step b1) is carried out in propanol, particularly 2-propanol. A volume of solvent of from 10 to 50 l, preferably 15 to 25 l, particularly preferably 17 to 19 l may be used per kilogram of the solvent-free extract. Preferably the filtrate from step b3) is concentrated by evaporation before the precipitation in step b4), particularly preferably to a dilution of 1:12 to 1:36 to the solid which is being formed or which is expected. The precipitation is preferably carried out by slowly cooling the filtrate to −15 to 15° C., preferably −5 to 15° C., particularly preferably 5 to 15° C. within a period of 5 to 50 h, preferably 5 to 20 h, particularly preferably 10 to 15 h. Then the crude betulinic acid may be separated from the mother liquor as a solid and dried, using methods known in the art (suction filter, centrifuge, decanter, pressure filter, filter dryer etc.). The crude betulinic acid thus obtained may still contain up to 15% solvent.

Preferably the filtering or suction composition described above consists of activated charcoal and/or kieselguhr.

Variable amounts of the filtering or suction composition may be used. The skilled man will be familiar with what are reasonable amounts for use as a filtering or suction composition in a purification step.

### TERMS AND DEFINITIONS

The term “betulinic acid” as used hereinbefore and hereinafter includes both betulinic acid as such and the hydrates and solvates thereof, preferably betulinic acid which has been solvated with 0.5 to 2 equivalents of a solvent. Within the scope of the invention all varieties of the genus Platanus are suitable. Planes which have proved particularly suitable are the American sycamore (Platanus occidentalis), the oriental plane (Platanus orientalis), Kerr's plane (Platanus kerrii), the Mexican plane (Platanus mexicana), the Californian sycamore (Platanus racemosa) and the Arizona sycamore (Platanus wrightii). However, subgenera of planes as well as hybrids derived from genera and subgenera such as the maple-leaf, common or London plane (Platanus×hispanica/acerifolia) (hybrid of P. occidentalis and P. orientalis) are also suitable as starting material for obtaining betulinic acid.

By the term “C₁₋₄-alcohol” are meant branched and unbranched alcohols with 1 to 4 carbon atoms and one or two hydroxy groups. Examples include: methanol, ethanol, n-propanol, 2-propanol, n-butanol, iso-butanol, sec-butanol or tert-butanol. In some cases the molecules mentioned above are also referred to by the abbreviations MeOH, EtOH, n-PrOH, 2-PrOH, n-BuOH, i-BuOH, t-BuOH, etc. Unless stated otherwise, the definitions propanol and butanol include all the possible isomeric forms of the groups in question. Thus for example propanol includes n-propanol and 2-propanol, butanol includes iso-butanol, sec-butanol and tert-butanol etc.

By the term “C₁₋₄-chloroalkanes” are meant branched and unbranched alkanes with 1 to 4 carbon atoms, which are substituted by one or more chlorine atoms. Examples of these include dichloromethane and chloroform.

By the term “C₃₋₆-ketones” are meant branched and unbranched alkanes with 3 to 6 carbon atoms, which are substituted by an oxo group at a non-terminal carbon atom. Examples of these include acetone, methylethylketone and methyl-2-butylketone.

The term xylene for the purposes of the invention may be a mixture of o-, m- and p-xylene, as well as optionally ethylbenzene. Theoretically, however, isomerically pure solvent may also be used.

### EXAMPLE

1A Preparation of Plane Bark Extract

To extract powdered plane bark on an industrial scale, a battery extraction apparatus is used consisting of three extractors, a pure distillate receiver and an evaporator. For fully loading the apparatus, 650 kg of powdered plane bark for each extractor are mixed with 260 l water in a mixer and introduced into the respective extractor using a conveyor belt. Each individual extractor is then topped up with 2.5 m³ of warm toluene/(+−)-2-pentanol/methanol mixture (88:7:5). Starting from the pure distillate receiver, toluene/(+−)-2-pentanol/methanol mixture (88:7:5) heated to 60° C. is pumped out through the three extractors connected in series at a flow rate of 2 m³/h. 2 m³ extract per hour are also fed into the evaporator from the system and concentrated therein by distilling off the solvent mixture.

The distillate obtained is freed from the water distilled off with it by means of a phase separator and the organic phase is then analysed by gas chromatography. If necessary, the composition of the organic phase is readjusted to 88:7:5 for toluene/(+−)-2-pentanol/methanol by the addition of fresh methanol or (+−)-2-pentanol. The toluene/(+−)-2-pentanol/methanol-mixture recycled in this manner is fed into the pure distillate receiver and its temperature is regulated to 60° C.

After 20 h the extraction process is stopped. The residual extract contained in the extractors is pumped out and also evaporated down in the evaporator. The total quantity of extract obtained is concentrated down to a volume of 1300 l. The content of total extract measured by determining the residue is 61 g/l.

1B Obtaining Betulinic Acid in Crude Form

1300 l of the extract obtained according to Example 1A are pumped from the evaporator into a distillation apparatus, then evaporated to dryness and the residue is suspended in 560 l of 2-propanol. It is evaporated to dryness again and then the distillation residue is refluxed with 1430 l of 2-propanol under normal pressure for 30 min. The suspension is combined with 11 kg kieselguhr and 24 kg activated charcoal and filtered hot through a pressure filter into a crystalliser and the distillation apparatus and the pressure filter are rinsed with 20 l of 2-propanol. 240 l of 2-propanol are distilled off from the 2-propanolic solution and then the contents of the crystalliser are cooled to 10° C. overnight. The resulting suspension is suction filtered, the solid is washed with 48 l of 2-propanol and then dried at 55° C. in the vacuum drying cupboard. The 2-propanol can be recovered by distillation.
43.4 kg of betulinic acid are obtained in crude form.

M.p. 309-310° C.; betulinic acid content (HPLC-UV): 83.9%; 2-propanol content (¹H NMR): 10.5%.

1C Obtainibg Betulinic Acid in Pure Form

43.4 kg crude betulinic acid obtained according to Example 1B are refluxed and dissolved in a distillation apparatus with 930 l dichloromethane and 325 l methanol. The mixture is left to cool to 30° C. and a suspension of 22 kg activated charcoal and 11 kg kieselguhr in 65 l methanol is added. The contents of the apparatus are again heated to boiling, filtered hot through a pressure filter into a crystalliser and the distillation apparatus and the pressure filter are rinsed with 50 l dichloromethane. 630 l of the filtrate are distilled off under normal pressure 630 l and the contents of the apparatus are then cooled to 5° C. overnight. The solid precipitated is separated off using a centrifuge, sprayed with 44 l of methanol at 5° C. and then dried at 50° C. in the vacuum drying cupboard. 28.9 kg of pure betulinic acid are obtained. M.p. 308-310° C.; betulinic acid content (HPLC-UV): 93.8%; dichloromethane content (¹H NMR): 2.1%; methanol content (¹H NMR): 2.3%.

1. A process for obtaining highly pure betulinic acid comprising extracting betulinic acid from ground plane bark and/or cortex, wherein the extraction is carried out with a solvent mixture consisting of toluene, xylene or toluene/xylene and
one or more C₁₋₆-alcohols, or
acetic acid esters, or
C₃₋₆-ketones.

2. The Process according to claim 1, wherein the plane bark is moistened with water before the extraction.

3. The Process according to claim 2, wherein the extraction is carried out continuously.

4. The Process according to claim 3, wherein the extraction is carried out at a temperature of 20 to 110° C.

5. The Process according to claim 1 comprising the following steps which are carried out successively to isolate the betulinic acid:
a) extracting plane bark in the solvent mixture;
b) removing the solvent mixture from the extract;
c) dissolving the residue in a suitable solvent; and
d) precipitating betulinic acid from the filtrate.

6. The Process according to claim 5, wherein between step b) and c) the following steps are carried out:
b1) suspending and dissolving the residue in propanol, butanol or a mixture thereof;
b2) mixing with a filtering or suction composition
b3) filtering the suspension
b4) precipitating the crude betulinic acid from the filtered extract.

7. The Process according to claim 6, wherein the filtering or suction composition consists of activated charcoal and/or kieselguhr.
